(19) United States
(12) Patent Application Publication
(10) Pub. No.: US 2021/0085265 A1
(43) Pub. Date: Mar. 25, 2021

(54) BED AND MEDICAL IMAGE DIAGNOSTIC DEVICE

(71) Applicant: Kengo NAKAHARA, Yamaguchi-shi (JP)

(72) Inventor: Kengo NAKAHARA, Yamaguchi-shi (JP)

(21) Appl. No.: 16/630,571

(22) PCT Filed: Oct. 27, 2017
(86) PCT No.: PCT/JP2017/039003

§ 371 Date: Jan. 13, 2020

(30) Foreign Application Priority Data
Jul. 19, 2017 (JP) 2017-140399

(51) Int. Cl. A61B 6/04; A61B 6/00

(52) U.S. Cl. CPC A61B 6/0407; A61B 6/487; A61B 6/0485; A61B 6/0478; A61B 6/54

(57) ABSTRACT

A bed capable of reducing a burden of posture conversion of a subject. There is provided a bed on which a subject is placed including: an upper body-holding bed configured to rotate around a rotational axis extending along a central axis of the subject, and to hold and rotate the upper body of the subject; and a lower body-holding bed configured to rotate around the rotational axis, and to hold and rotate the lower body of the subject, wherein the upper body-holding bed and the lower body-holding bed are configured so that one of these components rotates in accordance with rotation of the other.

### CROSS-REFERENCE TO RELATED APPLICATIONS

The present application claims priority under 35 U.S.C. § 119 to International Application No. PCT/JP2017/039003, filed Oct. 27, 2017. The contents of this application are incorporated herein by reference in their entirety. The present invention relates to a bed and a medical image diagnostic device including the bed.

### TECHNICAL FIELD

### BACKGROUND ART

It is considered that a bed on which a subject is placed and the posture of the subject is variably driven is widely used in the medical field typified by a medical image diagnosis device and the entertainment field such as a playground equipment, and can be utilized in the future. Hereinafter, although it is only an example, a description will be given by focusing on the “medical image diagnosis device” as “subject” =“examinee (subject)”.

In Patent Document 1, a subject holding device is disclosed, which comprises two rotation support shafts having support members facing each other with the subject sandwiched provided on the height axis of the subject in an X-ray fluoroscopic imaging device, and a non-stretchable cloth body covering the hammock-like non-stretchable cloth body with the upper part opened between the support members and partially covering and tightening the open upper part, and in which the two rotating spindles are synchronously rotated.

Meanwhile, in gastric cancer screening in Japan, there has been a history of performing X-ray imaging examination using barium as a main screening mean for almost half a century.

Until the 1990s, a large amount of low-concentration sol preparation barium was taken together with a small amount of foaming agent, and a photographing method mainly includes a “filling image” for photographing a portion where barium has accumulated at a semi-standing position or a standing position by performing a 180 degree posture conversion on an X-ray fluoroscopic imaging table. In the 2000s, along with the development of high-concentration and low-viscosity barium preparations, a great deal of “double contrast methods” has been introduced which focusing on the depiction of the gastric mucosa by using many foaming agents in combination with high-concentration barium.

In view of this, a new imaging method has been examined in The Japanese Society of Gastrointestinal Cancer Screening (see, for example, Non-Patent Document 1). The examined new imaging method is an imaging method mainly based on double contrast images. The subject drinks a foaming agent that is a negative contrast agent with water, while advancing the gastric lumen with the generated air, a barium sulfate preparation (positive contrast agent) that is a fluid inside the stomach taken by the subject moves inside the stomach. Since it is necessary for the barium sulfate preparation to adhere all over the gastric mucosa, the subject stands on the bed of the fluoroscopy device in a standing position, then becomes horizontal and starts an examination on the fluoroscopic imaging table. Right after this, a “360 degree right 3 times rotation in horizontal position” that has not been carried out before is performed immediately. Further, in order to improve mucosal visualization, it became necessary more than before to change the position several times during the examination. After the proper posture is obtained, a radiologist captures a prescribed number of stomach X-ray images. This imaging procedure dramatically improves image accuracy and contributes to the discovery of early gastric cancer for lifesaving.

### PRIOR ART DOCUMENTS

### Patent Document

[Patent Document 1] Japanese Utility Model Application Laid-open Publication No. Sho 54-50968 Non-Patent Document

[Non-Patent Document 1] The Japanese Society of Gastrointestinal Cancer Screening (edit), Stomach Cancer Screening Quality Management Committee (edit), “New Gastrography Guideline Revised Edition (2011)”, Igaku-Shoin, March 2011.

### SUMMARY OF THE INVENTION

### Problems to be Solved by Invention

Normally, in a gastrointestinal examination employing an X-ray fluoroscopic imaging device, a subject may suffer while converting the posture regarding the physical ability of the subject. It is understood that the same problem occurs when applied to, for instance, a playground equipment or the like.

The present invention has been made in view of the above-described circumstances, and an object thereof is to provide a bed capable of reducing a burden of posture conversion of a subject.

### Means for Solving Problems

According to the present invention, there is provided a bed on which a subject is placed comprising: an upper body-holding bed configured to rotate around a rotational axis extending along a central axis of the subject, and to hold and rotate the upper body of the subject; and a lower body-holding bed configured to rotate around the rotational axis, and to hold and rotate the lower body of the subject, wherein the upper body-holding bed and the lower body-holding bed are configured so that one of these components rotates in accordance with rotation of the other.

The bed according to the present invention includes the upper body-holding bed and the lower body-holding bed. The upper body-holding bed is configured to rotate around the rotational axis extending along the central axis of the subject, and to hold and rotate the upper body of the subject; and the lower body-holding bed is configured to rotate around the rotational axis, and to hold and rotate the lower body of the subject, especially the upper body-holding bed and the lower body-holding bed are configured so that one of these components rotates in accordance with rotation of the other. With such a configuration, the burden of the posture conversion of the subject can be reduced as compared with the related art.

Hereinafter, various embodiments of the present invention will be described below. These embodiments can be combined with each other.

Preferably, the bed further comprises a drive control unit configured to perform drive control so that one of the upper body-holding bed and the lower body-holding bed rotates in accordance with rotation of the other. Preferably, the bed further comprises a manual rotation unit through which the upper body-holding bed and the lower body-holding bed are configured so that one of these components rotates in accordance with rotation of the other of the components.

Preferably, the bed is configured to be movable from an upright state to a lying down state. Preferably, the bed further comprises: a lifting platform configured such that the lower body-holding bed can be placed with the subject standing upright in the upright state; and a holding chair configured to be fixed to the lifting platform, wherein the subject can sit in the upright state, and the lower limbs of the subject are held by the holding chair in the lying down state.

Preferably, the lower body-holding bed further comprises a lifting mechanism configured to lift and lower the lifting platform.

Preferably, the bed further comprises: a shoulder rest unit provided on the upper body-holding bed to press a shoulder of the subject; a grip unit provided to be capable of being gripped by the subject; and an upper limb accommodation unit configured to accommodate the upper limbs of the subject holding the grip unit.

Preferably, the bed further comprises a first airbag provided on the upper body-holding bed so as to inflate between the body of the subject and the upper body-holding bed.

Preferably, the bed further comprises a second airbag provided on the lower body-holding bed so as to inflate between the body of the subject and the lower body-holding bed.

Preferably, the bed further comprises a plurality of retraction grooves and a discharge hole. Defining a side on which the subject is placed on the bed as an inner side and defining the opposite side as an outer side, a plurality of retraction grooves provided on the inner side, and the discharge hole provided on the inner side of the retraction groove and configured to penetrate to the outer side.

Preferably, the lower body-holding bed has a double structure including an inner cylinder and an outer cylinder, only the inner cylinder rotates, and the outer cylinder is configured to be slidable in the rotation axis direction with respect to the inner cylinder.

Preferably, a medical image diagnostic device comprises a bed on which a subject is placed including an upper body-holding bed configured to rotate around a rotational axis extending along a central axis of the subject, and to hold and rotate the upper body of the subject; and a lower body-holding bed configured to rotate around the rotational axis, and to hold and rotate the lower body of the subject, wherein the upper body-holding bed and the lower body-holding bed are configured so that one of these components rotates in accordance with rotation of the other.

Preferably, the device further comprises an X-ray generation unit configured to generate X-rays; and an X-ray detection unit that detects X-rays; and the bed further comprises a central bed disposed between the X-ray generation unit and the X-ray detection unit.

### BRIEF DESCRIPTION OF DRAWINGS

FIG. 1 is an external view illustrating an upright state in which a bed unit included in an X-ray fluoroscopic imaging device according to an embodiment of the present invention.

FIG. 2 is an explanatory view illustrating an A-A cross-section of FIG. 1.

FIG. 3 is an external view illustrating a lying down state in which the bed unit included in the same X-ray fluoroscopic imaging device.

FIGS. 4A-4C are explanatory views illustrating a state of a subject to be inspected by the same X-ray fluoroscopic imaging device, and showing states of a prone position, a supine position, and a lateral position of the subject, respectively.

FIGS. 5A and 5B are a front view and a side view illustrating an upper body-holding included in the same X-ray fluoroscopic imaging device, respectively.

FIGS. 6A and 6B are schematic views illustrating a lifting mode of a lifting platform, respectively.

FIG. 7 is a schematic view illustrating a modification of the upper body-holding bed (or the lower body-holding bed).

FIG. 8 is a schematic view illustrating a modification of the bed unit.

FIG. 9 is a schematic view illustrating a modification of the lower body-holding bed related to FIG. 8.

### DESCRIPTION OF PREFERRED EMBODIMENTS

Hereinafter, embodiments of the present invention will be described with reference to the accompanying drawings to provide an understanding of the present invention. It should be noted that in the drawings, illustration of portions unrelated to the description may be omitted in some cases.

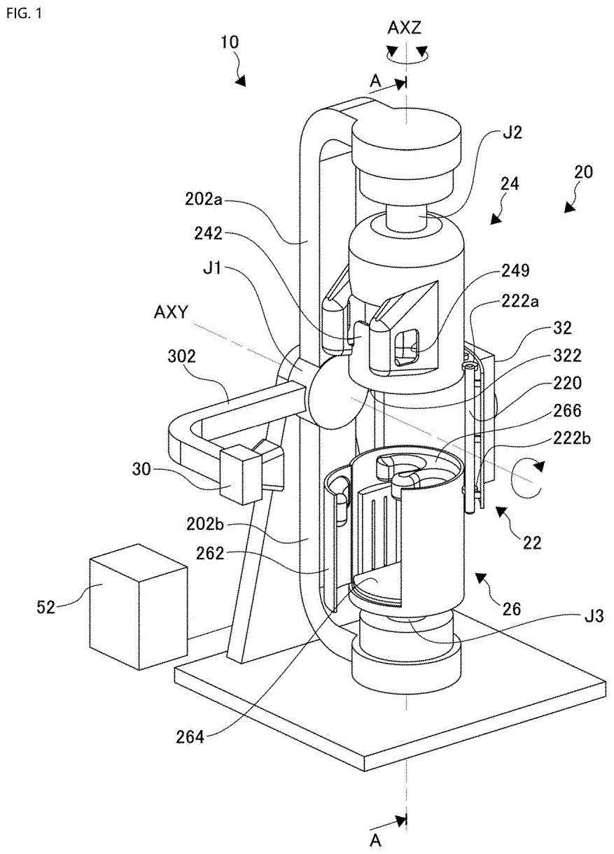

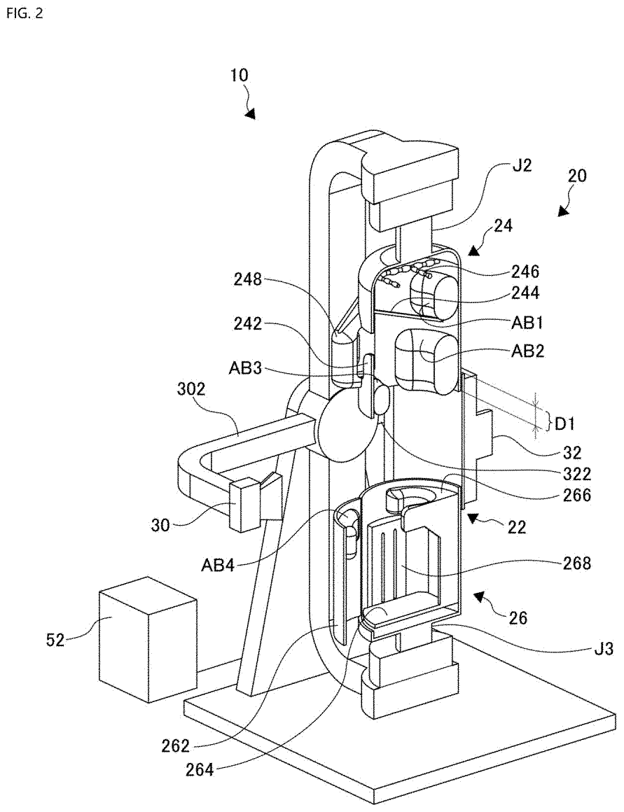

As shown in FIGS. 1 and 2, an X-ray fluoroscopic imaging device (an example of a medical image diagnostic device) 10 according to an embodiment of the present invention includes a bed unit 20 (an example of a “bed” in claims) and a drive control unit that controls driving of the bed unit 20.

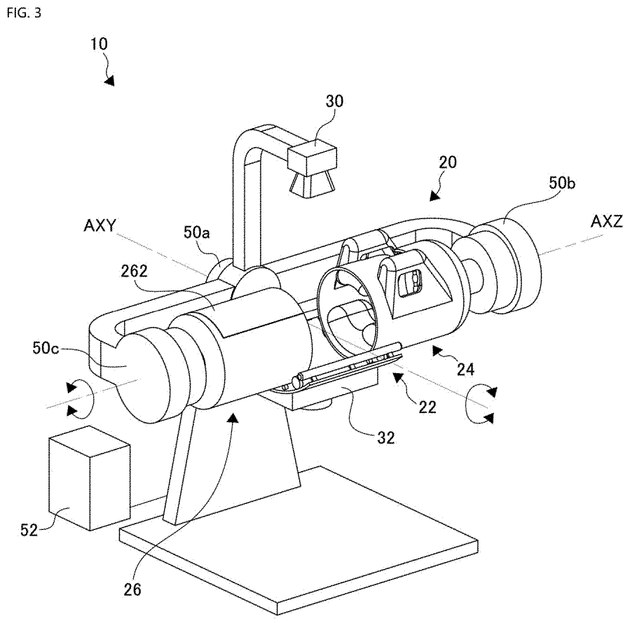

The bed unit 20 is supported by support arms 202a and 202b extending from a first shaft unit J1, and rotates around a rotation axis AXY together with an X-ray generation unit 30 and an X-ray detection unit 32. Therefore, by rotating around the rotation axis AXY, the bed unit 20 can move into a lying down state (see FIG. 3) from an upright state (see FIG. 1).

The X-ray generation unit 30 is supported by a support arm 302 extending from the first shaft unit J1, and can generate X-rays. The X-ray detection unit 32 is supported by a support arm 322 extending from the first shaft unit J1, and can detect X-rays generated by the X-ray generation unit 30. The X-ray detection unit 32 is, for example, a flat panel detector (FPD). However, the X-ray detection unit 32 may be arbitrary, and another example is image intensifier (I.I.).

The bed unit 20 includes a central bed 22, an upper body-holding bed 24, and a lower body-holding bed 26.

The central bed 22 is supported by the support arm 322 extending from the first shaft unit J1, and is disposed between the X-ray generation unit 30 and the X-ray detection unit 32. Further, the central bed 22 is a bed having a curved surface, and is disposed at a position that can support the waist or the abdomen of the subject when the bed unit 20 is in the lying down state. On both sides of the central bed 22, round bar-shaped handrails 220 extending in the longitudinal direction are provided.

In addition, the surface of the central bed 22 may be flat.

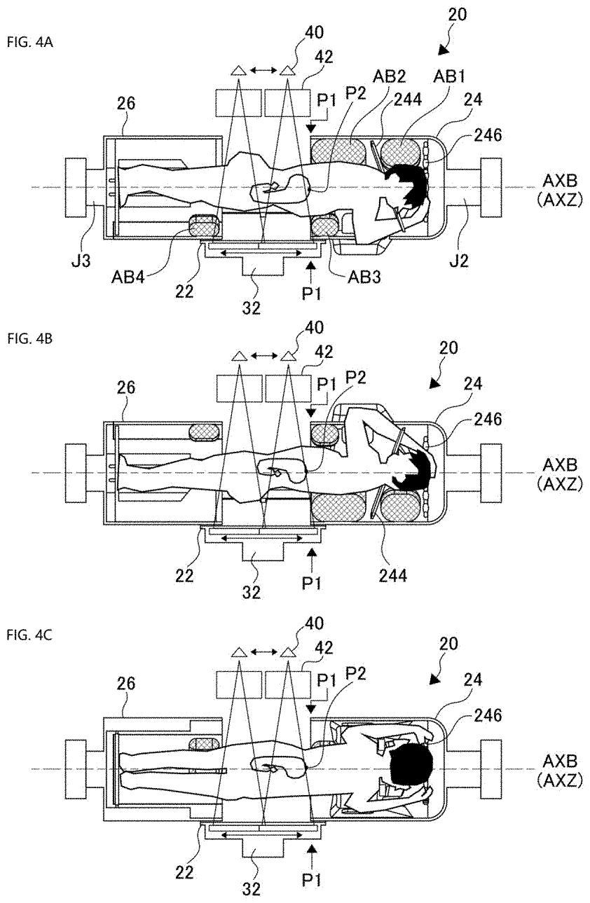

The upper body-holding bed 24 has a cylindrical shape and accommodate an upper body (from the chest to the head) of the subject inside (see FIGS. 4A to 4C). The upper body-holding bed 24 is supported by a plurality of rollers 222a provided on the central bed 22 and a second shaft unit J2 provided at a distal end portion of the support arm 202a extending from the first shaft unit J1, and can rotate around a rotation axis AXZ that intersects the rotation axis AXY and extends along a central axis AXB (see FIGS. 4A to 4C) of the body of the subject.

As shown by a part D1 in FIG. 2, an end portion of the upper body-holding bed 24 overlaps with an end portion of the central bed 22. As shown in FIGS. 4A to 4C, a position P1 in the direction in which the rotation axis AXY of an end edge of the upper body-holding bed 24 extends, is set to be closer to the head than a position P2 of the upper edge of the stomach buttock when viewed from the front side (viewed from the side of the X-ray generation unit 30), so as not to interrupt the detection of the X-rays passing through the stomach from a X-ray tube focal spot 40 of the X-ray generation unit 30 via a collimator 42.

As shown in FIG. 2, the upper body-holding bed 24 has a cheek rest unit 242, a shoulder rest unit 244, a grip unit 246, and an upper limb accommodation unit 248 therein.

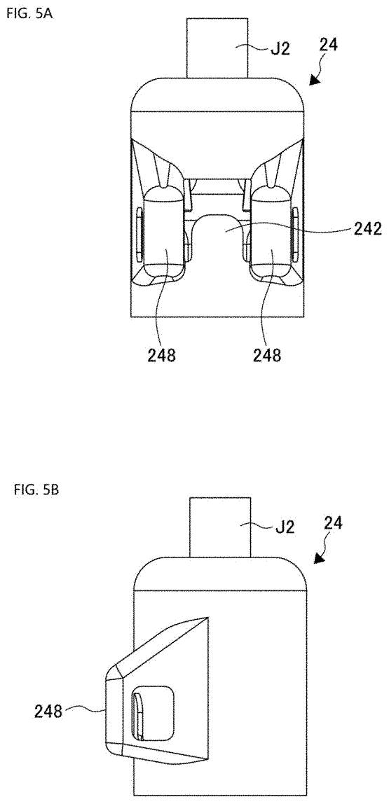

The cheek rest unit 242 (see FIG. 5A) can support a cheek of the subject. The area around the cheek rest is open, and a visual field of the subject is secured.

The shoulder rest unit 244 (see FIG. 2) can move in the direction in which the rotation axis AXY extends, and can be pressed against a shoulder of the subject. The shoulder rest unit 244 is provided with a detector (not shown) that detects contact with the shoulder.

The grip unit 246 is disposed to be closer to the second shaft unit J2 side than the head of the subject, and the upper body is stably held when the subject grips the grip unit 246. The upper limb accommodation unit 248 accommodates a part of upper limbs and a forearm of the subject who holds the grip unit 246. A window 249 (see FIG. 1) is provided on a side surface of the upper limb accommodation unit 248.

Inside the upper body-holding bed 24, an LED light (not shown) serving as an illumination, a monitoring camera (not shown) for the radiologist to grasp the state of the subject, and an interphone (not shown) for the subject to talk with the radiologist are provided.

An elastic cushioning member (not shown) is provided on the inner side of the upper body-holding bed 24 in order to reduce an impact generated by the body of the subject slipping and colliding when the upper body-holding bed 24 rotates.

In addition, as shown in FIGS. 4A to 4C, a plurality of first airbags AB1 to AB3 that inflate and deflate between the body of the subject and the upper body-holding bed 24 are provided on the inner side of the upper body-holding bed 24, and an air pump (not shown) is connected to the first airbags AB1 to AB3. Each of the first airbags AB1 to AB3 can inflate and deflate individually. The first airbags AB1 to AB3 inflate when the upper body-holding bed 24 rotates, so that the displacement of the body of the subject is reduced, and the upper body is stably held. It should be noted that an airbag may be further provided on the cheek rest unit 242.

As shown in FIG. 1, the lower body-holding bed 26 has a cylindrical shape, and the lower body (from the thigh to the toes) of the subject is accommodated therein. The lower body-holding bed 26 is supported by a plurality of rollers 222b provided on the central bed 22 and a third shaft unit J3 provided at a distal end portion of the support arm 202b extending from the first shaft unit J1, and can rotate around a rotation axis AXZ that intersects the rotation axis AXY and extends along the central axis AXB (see FIGS. 4A to 4C) of the body of the subject.

As shown in FIG. 2, the lower body-holding bed 26 includes an opening door 262, a lifting platform 264, and a holding chair 266.

The opening door 262 is a door for the subject to enter when the bed unit 20 is in the upright state (see FIG. 1), and is provided on the front side (the side of the X-ray generation unit 30). The lower body-holding bed 26 is provided with a detector (not shown) for detecting that the opening door 262 is open. Further, a lock mechanism (not shown) for locking the opening door 262 is provided on the lower body-holding bed 26.

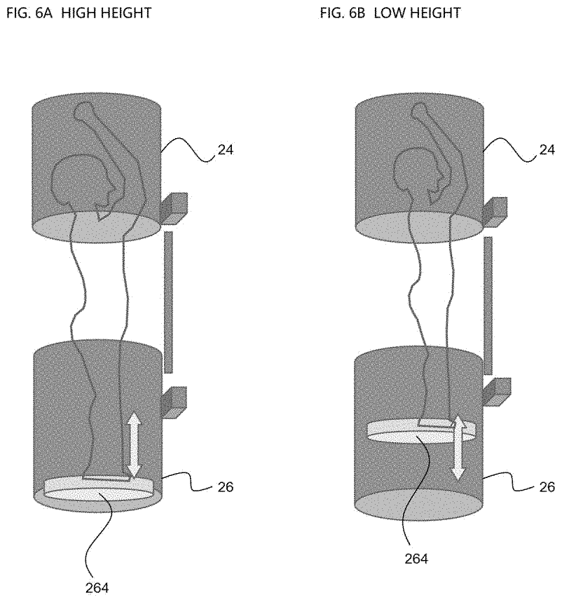

The lifting platform 264 is a platform on which the subject stands. As shown in FIG. 2, the lifting platform 264 is driven by a lifting mechanism 268, and can lift and lower (see FIGS. 6A and 6B) when the bed unit 20 is in the upright state (see FIG. 1). That is, the lifting platform 264 can be adjusted to an appropriate height in accordance with the height of the subject. The lifting mechanism 268 includes a motor (not shown), and can lift and lower the lifting platform 264 by transmitting the power of the motor using, for example, a chain (not shown). The power of the motor is supplied via a brush (not shown) provided inside the third shaft unit J3.

The holding chair 266 is fixed to the lifting platform 264, and the subject can sit down when the bed unit 20 is in the upright state (see FIG. 1).

The holding chair 266 is formed with two notches into which both the legs of the subject are inserted. The holding chair 266 is disposed at a position where the abdomen side is held rather than both knees of the subject when the bed unit 20 is in a lying down state (see FIG. 3). Therefore, since the lower leg is held by the holding chair 266, the lower body is stably held when the lower body-holding bed 26 rotates.

As shown in FIGS. 4A to 4C, a second airbag AB4 that inflates and deflates between the body of the subject and the lower body-holding bed 26 is provided on the inner side of the lower body-holding bed 26, and an air pump (not shown) is connected to the second airbag AB4. The second airbag AB4 can inflate and deflate independently of the first airbags AB1 to AB3. When the second airbag AB4 inflates while the lower body-holding bed 26 rotates, the impact generated by the body of the subject slipping and colliding is reduced, and the lower body is stably held.

As shown in FIG. 3, the drive control unit includes a first drive mechanism 50a, a second drive mechanism 50b, a third drive mechanism 50c, and a control device 52.

The first drive mechanism 50a includes a motor (not shown), and can rotate the bed unit 20 around the rotation axis AXY. The second drive mechanism 50b includes a motor (not shown), and can rotate the upper body-holding bed 24 around the rotation axis AXZ. The second drive mechanism 50b may be a power transmission mechanism that transmits the power of the motor by using a chain. The third drive mechanism 50c includes a motor (not shown), and can rotate the lower body-holding bed 26 around the rotation axis AXZ. The third drive mechanism 50c may be a power transmission mechanism that transmits the power of the motor by using a chain. The control device 52 can control the motors of the first drive mechanism 50a, the second drive mechanism 50b, and the third drive mechanism 50c, respectively. The control device 52 operates each motor based on the following interlock conditions 1 to 3.

(Interlocking condition 1) In a state in which the opening door 262 is opened, the subject does not rotate the upper body-holding bed 24 and the lower body-holding bed 26 beyond 45 degrees left-right from the upright front state).

(Interlocking condition 2) In a state in which the opening door 262 is opened, the bed unit 20 is not moved between the upright state (see FIG. 1) and the lying down state (see FIG. 3).

(Interlocking condition 3) When the shoulder rest unit 244 is not in contact with the shoulder, the bed unit 20 is not moved at the reverse tilt (head lower than or equal to the horizontal −5 degrees).

Therefore, by controlling these motors, the drive control unit can perform drive control so that one of the upper body-holding bed 24 and the lower body-holding bed 26 rotates in accordance with rotation of the other one of the upper body-holding bed 24 and the lower body-holding bed 26. Preferably, the upper body-holding bed 24 and the lower body-holding bed 26 rotate at the same time, but may be rotated with a slight time difference, either intentionally or unintentionally.

Note that the above-described drive control unit may simply be a movable unit. In other words, instead of the first drive mechanism 50a, the second drive mechanism 50b, and the third drive mechanism 50c, a simple rotation mechanism without a motor (that is, a first rotation mechanism 50a, a second rotation mechanism 50b, and a third rotation mechanism 50c) may be employed. In such a case, the radiologist or the like can appropriately rotate the constituent elements (refer to the above description) related to the first to third rotation mechanisms 50a to 50c via a manual rotation unit (for example, a handle or a steering wheel).

Further, the drive control unit may separately provide the manual rotation unit in consideration of emergency or the like with inventive approach. In other words, although the drive control is normally performed by the drive control unit, if any trouble occurs, the radiologist or the like can manually rotate the constituent elements (see the above description) related to the first to third drive mechanisms 50a to 50c as appropriate through the manual rotation unit instead.

Next, an X-ray fluoroscopic imaging method using the X-ray fluoroscopic imaging device 10 will be described. The X-ray fluoroscopic imaging method is performed in accordance with the following steps. It should be note that if possible, the following steps may be executed in reverse order or in parallel. In addition, the operation of the X-ray fluoroscopic imaging device 10 in each step is performed based on the operation of the radiologist.

(Step S1)

The bed unit 20 is in the upright state as shown in FIG. 1, and the subject enters the lower body-holding bed 26 by opening the opening door 262. The subject stands on the lifting platform 264 with the back facing the central bed 22.

(Step S2)

The subject sits on the holding chair 266 and drinks barium in the cup, then performs an esophageal photography. The cup is placed in a cup holder (not shown).

(Step S3)

After the esophageal photography is completed, the subject returns the cup to the cup holder and closes the opening door 262. The opening door 262 is locked by a lock mechanism (not shown). The subject stands on the lifting platform 264 in a state in which lower limbs are inserted into the notches of the holding chair 266. The shoulder rest unit 244 (see FIG. 2) is lowered to press the shoulder (trapezius) of the subject. When the shoulder rest unit 244 cannot be brought into contact with the shoulder, the lifting platform 264 is raised. The shoulder rest unit 244 and the lifting platform 264 prevent the body from being displaced in the direction in which the rotation axis AXZ extends.

(Step S4)

An air pump (not shown) operates to inflate the first airbag AB1 to AB3 and the second airbag AB4. When the subject holds the grip unit 246 with both hands in a state where the upper limbs of the subject are accommodated by the upper limb accommodation unit 248 (see FIGS. 4A to 4C).

(Step S5)

The bed unit 20 turns into a lying down state as shown in FIG. 3.

(Step S6)

The drive control unit performs drive control so that the upper body-holding bed 24 and the lower body-holding bed 26 rotate in synchronization. That is, the drive control unit performs drive control so that the lower body-holding bed 26 rotates in accordance with the rotation of the upper body-holding bed 24. In addition, the drive control unit may perform drive control so that the upper body-holding bed 24 rotates in accordance with the rotation of the lower body-holding bed 26. By rotating the upper body-holding bed 24 and the lower body-holding bed 26, the posture conversion of the subject is assisted.

(Step S7)

An X-ray imaging is performed at a desired rotational position (appropriate posture). However, the first airbag located on the side of the central bed 22 of the first airbags AB1 to AB3 automatically deflates according to the rotational position. When the first airbag located on the side of the central bed 22 deflates, the body approaches the X-ray detection unit 32, and the image is prevented from being enlarged and blurred. Further, according to the rotational position, the first airbag besides ones located on the side of the central bed 22 is automatically inflated. When the first airbag is automatically inflated, the impact caused by the rotation is suppressed.

(Step S8)

The bed unit 20 rotates from the lying down state (see FIG. 3) to the upright state (see FIG. 1).

(Step S9)

After the compression photography is performed in a standing position, the lifting platform 264 on which the subject is placed descends. The opening door 262 is opened, the subject gets off the bed unit 20 and the examination ends.

As described above, according to the X-ray fluoroscopic imaging device 10 according to the present embodiment, since the upper body-holding bed 24 and the lower body-holding bed 26 rotate while holding the upper body and lower body of the subject, the burden of the subject to convert the posture by his/her own force is reduced. Note that the X-ray fluoroscopic imaging device 10 may be configured such that when the size or operation of the bed unit 20 does not match the physique or personal characteristics of the subject, the subject can perform posture conversion (examination) such as prone position.

Although the embodiments of the present invention have been described above, the present invention is not limited to the embodiments described above, and all changes and the like that do not depart from the gist thereof are within the scope of the present invention. In particular, the bed according to the present invention can be implemented by the following aspects.

The positions and sizes of the first airbags AB1 to AB3 and the second airbag AB4 are not limited to the above-described embodiments.

The bed unit 20 is not limited to the application to the medical image diagnosis device, and may be applied to, for example, a playground equipment in an amusement park. [0056]

The medical image diagnostic device is not limited to the X-ray fluoroscopic device. Other examples of the medical image diagnostic device include an ultrasonic diagnostic device comprising a bed unit 20, an X-ray CT device, and a blood vessel X-ray imaging device. Furthermore, since examines such as CT Colonography (CTC), CT Ulography (CTU), Colonoscopy, Abdominal Ultrasonography, Heart Ultrasound Examination, Barium Enama X-ray Inspection, and Myelography include the up and down movement and rotation of the subject, the pain of the subject can be expected to be reduced by adopting the bed unit 20 according to the present invention as these examination devices.

In the present embodiment, the bed unit 20 is configured to be able to move from the upright state (see FIG. 1) to the lying down state (see FIG. 3), but this may not necessarily be adopted. That is, the bed unit 20 may be implemented so that the bed unit 20 is always lying down. In such a case, as a matter of course, the first rotation drive mechanism 50a is not necessary.

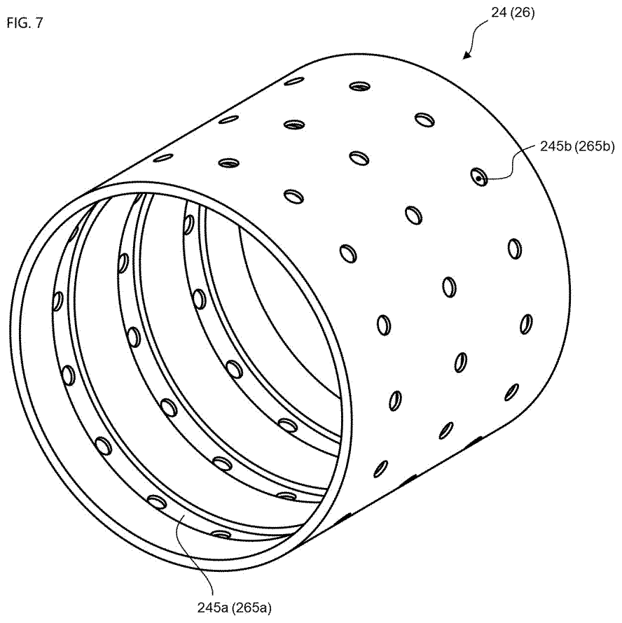

On the bed unit 20 during the examination, there is a slight possibility that a liquid such as a vomiting substance like barium, blood, or excrement (urine/feces) flows out from the body of the subject and contaminate the bed unit 20. In such a case, for example, in order to prevent electrical components such as the first to third drive mechanisms 50a to 50c and the lifting mechanism 268 from contacting with the liquid, liquid retract discharging units 245 and 265 may be provided on the inner wall of the bed unit 20 (an example of “inner side” in the claims) (see FIG. 7). This will be described in detail.

The upper body-holding bed 24 comprises the liquid retract discharging unit 245, which includes a retraction groove 245a and a discharge hole 245b. Similarly, the lower body-holding bed 26 includes the liquid retract discharging unit 265, which is composed of a retraction groove 265a and a discharge hole 265b. It should also be noted that the liquid retract discharging units 245 and 265 are provided in gaps between the airbags (the first and second airbags AB1 to AB4). Moreover, the present invention may implement by providing either one of the liquid retract discharging units 245 and 265.

As shown in FIG. 7, the retraction groove 245a (265a) is provided so as to extend along the inner wall of the upper body-holding bed 24 with a cylindrical shape. In the unlikely event that the liquid as described above is discharged from the subject, the liquid can flow into the retraction groove 245a (265a), thereby suppressing the liquid from spreading over a wide range (advantageous effect). In addition, although three retraction grooves 245a are provided in FIG. 7, the number of retraction grooves 245a is not limited thereto.

Further, inside the retraction groove 245a (265a) (especially the bottom surface portion of the groove), a plurality of discharge holes 245b (265b) are provided so as to annularly surround the plurality of the discharge holes 245b (265b). The discharge hole 245b (265b) penetrates to the outside of the bed unit 20, and can quickly discharge the liquid discharged from the subject to the outside of the bed unit 20 (advantageous effect). The number of the discharge holes 245b (265b) is not particularly limited.

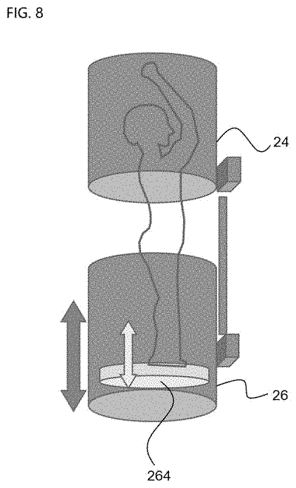

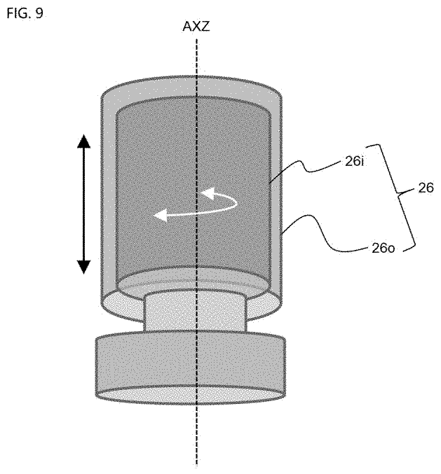

In the present embodiment, the lifting platform 264 is driven by the lifting mechanism 268 and can be lifted and lowered in accordance with the height of the subject. However, when the distance between the upper body-holding bed 24 and the lower body-holding bed 26 remains constant, there is a concern that the holding of the lower body (pelvis or the like) of the subject having a low height becomes unstable. Therefore, the lower body-holding bed 26 may be configured to be able to slide in the rotation axis AXZ direction (the height direction of the subject) so that the lower body can be held more safely even if the subject has a low height(See FIGS. 6B and 8). For example, as shown in FIG. 9, the lower body-holding bed 26 may have a double structure including an inner cylinder 26i and an outer cylinder 26o. In this case, only the inner cylinder 26i rotates around the rotation axis AXZ, and the outer cylinder 26i is configured to be slidable in the direction of the rotation axis AXZ (the height direction of the subject) with respect to the inner cylinder 26i. By adopting such a configuration, it is possible to appropriately adjust the position of the lower body-holding bed 26 even for a subject having a low height, and to hold the lower body of the subject more safely (advantageous effect).

While certain embodiments and modifications thereof according to the present invention have been described, these embodiments are presented as examples and are not intended to limit the scope of the invention. These novel embodiments can be implemented in various other forms, and various omissions, replacements, and changes can be apparent in light of the foregoing description. The accompanying claims and their equivalents are intended to cover such forms or modifications as would fall within the scope and spirit of the invention.

### REFERENCE SIGNS LIST

10 X-ray fluoroscopic device
20 Bed unit
202a, 202b Support arm
22 Central bed
222a, 222b Roller
24 Upper body-holding bed
242 Cheek rest unit
244 Shoulder rest unit
245 Liquid retract discharging unit
245a Retraction groove
245b Discharge hole
246 Grip unit
248 Upper limb accommodation unit
249 Window
26 Lower body-holding bed
26i Inner cylinder
26o Outer cylinder
262 Opening door
264 Lifting platform
265 Liquid extract exclusion unit
265a Extract groove
265b Discharge hole
266 Holding chair
268 Lifting mechanism
30 X-ray generation unit
302, 322 Support arm
32 X-ray detection unit
40 X-ray tube focal spot
42 Collimator
50a First drive mechanism (first rotation mechanism)
50b Second drive mechanism (second rotation mechanism)
50c Third drive mechanism (third rotation mechanism)
52 Control device
J1 First shaft unit
J2 Second shaft unit
J3 Third shaft unit
AB1-AB3 First airbag
AB4 Second airbag

1. A bed on which a subject is placed comprising:
an upper body-holding bed configured to rotate around a rotational axis extending along a central axis of the subject, and to hold and rotate the upper body of the subject; and
a lower body-holding bed configured to rotate around the rotational axis, and to hold and rotate the lower body of the subject,
wherein the upper body-holding bed and the lower body-holding bed are configured so that one of these components rotates in accordance with rotation of the other.

2. The bed according to claim 1, further comprising a drive control unit configured to perform drive control so that one of the upper body-holding bed and the lower body-holding bed rotates in accordance with rotation of the other.

3. The bed according to claim 1, further comprising a manual rotation unit through which the upper body-holding bed and the lower body-holding bed are configured so that one of these components rotates in accordance with rotation of the other.

4. The bed according to claim 1, wherein the bed is configured to be movable from an upright state to a lying down state.

5. The bed according to claim 4, further comprising:
a lifting platform configured such that the lower body-holding bed can be placed with the subject standing upright in the upright state; and
a holding chair configured to be fixed to the lifting platform, wherein the subject can sit in the upright state, and lower limbs of the subject are held by the holding chair in the lying down state.

6. The bed according to claim 5, wherein the lower body-holding bed further comprises a lifting mechanism configured to lift and lower the lifting platform.

7. The bed according to claim 1, further comprising:
a shoulder rest unit provided on the upper body-holding bed to press a shoulder of the subject;
a grip unit provided to be capable of being gripped by the subject; and
an upper limb accommodation unit configured to accommodate upper limbs of the subject holding the grip unit.

8. The bed according to claim 1, further comprising:
a first airbag provided on the upper body-holding bed so as to inflate between the body of the subject and the upper body-holding bed.

9. The bed according to claim 1, further comprising:
a second airbag provided on the lower body-holding bed so as to inflate between the body of the subject and the lower body-holding bed.

10. The bed according to claim 1, further comprising:
defining a side on which the subject is placed on the bed as an inner side and defining the opposite side as an outer side, a plurality of retraction grooves provided on the inner side; and
a discharge hole provided on the inner side of the retraction groove and configured to penetrate to the outer side.

11. The bed according to claim 1, wherein the lower body-holding bed has a double structure including an inner cylinder and an outer cylinder, only the inner cylinder rotates, and the outer cylinder is configured to be slidable in the rotation axis direction with respect to the inner cylinder.

12. A medical image diagnostic device comprising:
a bed on which a subject is placed comprising:
an upper body-holding bed configured to rotate around a rotational axis extending along a central axis of the subject, and to hold and rotate the upper body of the subject; and
a lower body-holding bed configured to rotate around the rotational axis, and to hold and rotate the lower body of the subject,
wherein the upper body-holding bed and the lower body-holding bed are configured so that one of these components rotates in accordance with rotation of the other.

13. The medical image diagnostic device according to claim 12, further including:
an X-ray generation unit configured to generate X-rays; and
an X-ray detection unit configured to detect X-rays,
wherein the bed further comprises a central bed disposed between the X-ray generation unit and the X-ray detection unit.
